(19) United States
(12) Patent Application Publication
(10) Pub. No.: US 2025/0191696 A1
(43) Pub. Date: Jun. 12, 2025

(54) DNA DATA STORAGE DEVICE WITH VARIABLE RELIABILITY TIERS

(71) Applicant: Western Digital Technologies, Inc., San Jose, CA (US)

(72) Inventors: David Avraham, Even Yehuda (IL); Ran Zamir, Ramat Gan (IL); Alexander Bazarsky, Holon (IL)

(21) Appl. No.: 18/531,675

(22) Filed: Dec. 6, 2023

(51) Int. Cl. G16B 50/30; G06F 11/00; G11C 13/02; G16B 50/50

(52) U.S. Cl. CPC G16B 50/30; G06F 11/008; G11C 13/02; G16B 50/50

(57) ABSTRACT

A DNA-based storage system stores DNA strands that are divided into N tiers. Due to the nature of the DNA storage channel, certain types of errors occur less frequently toward the front of a DNA strand and occur more frequently near the end of the DNA strand. As such, each tier stores data having a different level of importance when compared with other types of data stored in the different tiers. Because data stored near the front of the DNA strand has fewer errors than data stored near the end of the DNA strand, the data stored near the front of the DNA strand is decoded more quickly when compared with data near the end of the DNA strand.

### BACKGROUND

DNA-based storage systems are emerging as a promising storage technology. DNA is a long molecule made up of four nucleotide bases—adenine (A), cytosine (C), thymine (T) and guanine (G). For storage purposes, base units (ACTG) of synthesized DNA can be used to encode information—similar to how a string of ones and zeros represent data in traditional electronic storage systems. The encoded information may then be stored, subsequently accessed and decoded.

For example, DNA-based storage systems typically store DNA data using three main processes—synthesis (or writing) in which the base units of synthesized DNA are joined together to produce a desired DNA strand; storage, in which the DNA strand is stored in a DNA-based storage medium; and sequencing (or reading), in which the DNA strand is translated to binary/digital data.

While DNA-based storage systems are more dense than traditional electronic data storage systems, DNA-based storage systems are more prone to errors. For example, during synthesis, storage and/or sequencing, various symbols in the DNA strand may be inserted or deleted (errors known as an “indels,” for “insertions” or “deletions”). In other examples, during synthesis, storage and/or sequencing, one symbol (or multiple symbols) in the DNA strand may be substituted for another symbol (errors known as “substitutions”).

One challenge with “indel” type errors is that any insertion or deletion can shift the data enough to generate high bit error rates (BER). For example, an alignment shift of the bit stream just one to the left or one to the right can cause the data to be unrecognizable over the original data.

However, indel type errors occur less frequently toward the front of a DNA strand and occur more frequently near the end of the DNA strand. Accordingly, when encoding and decoding the DNA strand, it would be beneficial to utilize this phenomenon to maximize storage utilization and ensure certain types of data are reliably stored.

### SUMMARY

The present application describes a DNA-based storage system that stores DNA strands having different reliability tiers. For example, a DNA strand is divided into N tiers and each tier stores data having a different level of importance (e.g., when compared with other types of data). Due to the nature of the DNA storage channel, indel type errors occur less frequently toward the front of a DNA strand and occur more frequently near the end of the DNA strand. Because data that is stored near the front of the DNA strand has fewer errors than data stored near the end of the DNA strand, the data stored near the front of the DNA strand is decoded more quickly when compared with data near the end of the DNA strand.

Thus, different types of data (or data having different levels of importance) are stored in different tiers. For example, a first tier of the DNA strand stores data that is used for storage management (e.g., data corresponding to headers, data indexing, address information, a strand identifier) and/or data that is used for a decoding process (e.g., data statistics, data associated with cyclical redundancy checks). The second tier of the DNA strand stores data that that is less important/critical (or has a different functionality) when compared with the data stored in first tier. The Nth tier stores data that is less important/critical when compared with the data that is stored in the first tier through the N−1 tier. For example, the data that is stored in the Nth tier may include data logs, additional parity information (e.g., parity information that is in addition to a minimum amount of parity information), additional cyclic redundancy check data and/or pointer data (e.g., information that points to the next DNA strand).

Accordingly, the present disclosure describes a DNA-based storage system that stores a DNA strand having a first reliability tier and a second reliability tier. In an example, the first reliability tier is associated with a first type of data and the second reliability tier is associated with a second type of data.

In another example, the present disclosure describes a DNA-based storage system that includes an encoding system. In an example, the encoding system is operable to receive data to be encoded on a DNA strand. The encoding system also identifies a first type of data in the received data and a second type of data in the received data. The encoding system also associates the first type of data with a first tier in the DNA strand and associates the second type of data with a second tier in the DNA strand.

The present disclosure also describes a DNA-based storage system that includes a means for encoding data on a DNA strand. In an example, the DNA strand includes a first reliability tier that is associated with a first type of data and a second reliability tier that is associated with a second type of data. The DNA-based storage system also includes means for storing the DNA strand and means for decoding the DNA strand.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following Figures.

FIG. 1 illustrates a data storage system according to an example.

FIG. 2 illustrates a DNA strand having N reliability tiers according to an example.

FIG. 3 illustrates multiple DNA strands that have the same data in later tiers but different data in the beginning tiers according to an example.

FIG. 4A illustrates how a first amount of parity information is provided to a first portion of a DNA strand during an error correction process according to an example.

FIG. 4B illustrates how a second overlapping portion is provided to a second portion of the DNA strand of FIG. 4A according to an example.

FIG. 5 illustrates a DNA strand being divided into one or more portions or sub-codes, with each portion or sub-code being associated with different amounts of parity information according to an example.

FIG. 6 illustrates a method for decoding a DNA strand according to an example.

FIG. 7 illustrates a method for encoding data for a DNA strand having different reliability tiers according to an example.

FIG. 8 is a block diagram of a system that includes a host device and a data storage device according to an example.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems or devices. Accordingly, examples may take the form of a hardware implementation, an entirely software implementation, or an implementation combining software and hardware aspects. The following detailed description is therefore not to be taken in a limiting sense, and the scope of the present disclosure is defined by the appended claims and their equivalents.

In a DNA storage channel, several different types of errors can occur during a synthesis process and a sequencing process. One example error type is a substitution error. In a substitution error, a base that was intended to be one particular DNA nucleotide (e.g., an ‘A’, ‘T’, ‘G’, or ‘C’) is swapped for one of the other nucleotides. Since most DNA encodings use these nucleotides to represent different binary values (e.g., ‘A’=00, ‘T’=01, ‘G’=10, ‘C’=11), a substitution of one nucleotide for another, if left uncorrected, may cause some of the data to be incorrectly recreated during the decoding process. However, such substitution errors are generally limited in scope to just the few bits that the erroneous base represents. Further, at least some such substitution errors can be addressed and corrected with some known error correction protocols.

In contrast, indel type errors (“inserts” and “deletes”) can cause more significant issues in the DNA storage channel. More specifically, insert and delete errors each involve either adding a base to, or deleting a base from, the DNA strand. Such indel errors induce a shift in the alignment of the remaining bases. For example, a single insert error can not only introduce two additional bits into the data stream, but because the DNA strand is an ordered structure of encoded data, this alignment shift of just one base or two bits shifts all of the other bits to the back by two places. Such misaligned data can cause the decoding operation to generate garbled data that may be useless to the end user.

As previously discussed, due to the nature of the DNA storage channel, indel type errors occur less frequently toward the front of the DNA strand and occur more frequently near the end of the DNA strand. Thus, a bit error rate (BER) after an indel point will be higher than a BER up to the indel point. As the number of errors in a DNA strand increase, it takes more time to correct the errors.

In order to address the above, the DNA-based storage system described herein stores DNA strands having different reliability tiers. For example, a DNA strand is divided into N tiers. In an example, each tier stores data having a different level of importance and/or provides different functionality (e.g., when compared with other types of data).

For example, a first tier of the DNA strand stores a first type of data. The first type of data may include data having a first type of functionality or data, that if decoded and/or error corrected as efficiently as possible, would increase the reliability and efficiency of the DNA-based data storage device. In an example, the first type of data is data that is used for storage management (e.g., data corresponding to headers, data indexing, address information, a strand identifier) and/or data that is used for a decoding process (e.g., data statistics, data associated with cyclic redundancy checks). The second tier of the DNA strand stores data that that is less critical or important when compared with the data stored in first tier. The Nth tier stores data that is less critical when compared with the data that is stored in the first tier through the N−1 tier. For example, the data that is stored in the Nth tier may include data logs, additional parity information (e.g., parity information that is in addition to a minimum amount of parity information), additional cyclic redundancy check data and/or pointer data (e.g., information that points to the next DNA strand).

Because data that is stored near the front of the DNA strand has fewer errors, this data is typically decodable more quickly when compared with data near the end of the DNA strand (that typically has more errors). Thus, data that is identified as important, essential or critical can be decoded more quickly when compared with data that is identified as less important, essential or critical.

In another example, data at the beginning of a DNA strand may require fewer copies when compared with data at the end of a DNA strand. Thus, multiple copies of a DNA strand have different data stored in the first tier or section while copies of the same data are stored in later tiers or sections.

Accordingly, the present application includes many technical benefits in the area of DNA storage and retrieval including, but not limited to, enabling denser storage by reducing the number of copies of data that require fewer error corrections during a decoding process, improving the organization and management of data in a DNA-based storage system and improving data utilization by ensuring important data is accessed and decoded with minimal error correction.

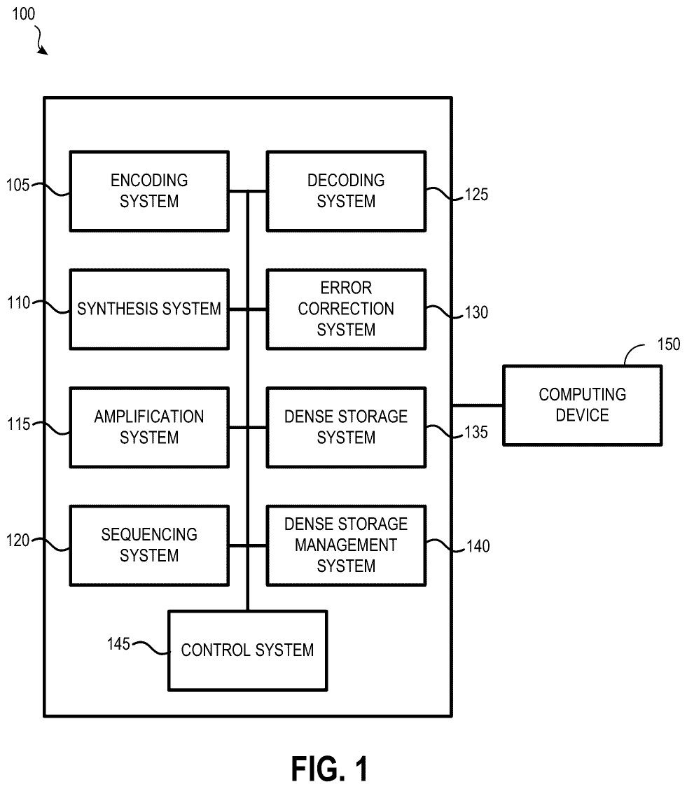

These various benefits and examples will be described in greater detail below with reference to FIG. 1-FIG. 8.

FIG. 1 illustrates a data storage system 100 according to an example. The data storage system 100 may be used to store data that is “more dense” when compared to data that is stored in a traditional electronic storage medium such as, for example, hard disks, optical disks, flash memory, and the like. For example, the data storage system 100 may be used to store synthetic DNA-based data.

DNA includes four naturally occurring nucleotide bases: adenine (A), cytosine (C), thymine (T) and guanine (G). In order to store data in synthetic DNA, received data is encoded to the various nucleotide bases. For example, data received as ones and zeros is encoded or otherwise mapped to various sequences of the synthetic DNA nucleotide bases. Once encoded, the data may be synthesized (e.g., written) and stored (e.g., in a dense storage system). To retrieve the stored data, the synthetic DNA molecules are sequenced (e.g., read) and subsequently decoded.

As part of the decoding process, the synthetic DNA nucleotide bases are remapped to the original ones and zeros. Each of these processes will be discussed in greater detail below. Effectively, the binary system of 0's and 1's (e.g., two states of a binary “base-2” numeral system, represented in one conventional binary bit) are represented in a quaternary system of A's, C's, T's, and G's (e.g., four states of a quaternary “base-4” numeral system, represented by a single nucleotide of DNA) when the source binary (e.g., base-2) data is encoded in the quaternary (e.g., base-4) system of nucleotides.

Although synthetic DNA-based data and associated DNA-based storage systems are specifically mentioned, the systems and methods described herein may be applicable to traditional electronic storage mediums/systems and/or traditional digital/binary data.

In an example, the data storage system 100 includes an encoding system 105. The encoding system 105 receives digital/binary information and/or data (e.g., binary ones and zeros, or “base-2”) from a computing device (e.g., computing device 150) or from another source. This data may be referred to herein as “input data,” “source data,” or “original data.” Such input is initially stored and represented in conventional base-2 binary (e.g., when not embodied in DNA). When the input data is received, the encoding system 105 converts or maps the ones and zeros of the original data into various DNA strands using the synthetic DNA nucleotide bases A, C, T, and G. For example, the DNA nucleotide base “A” may be assigned a value 00, the DNA nucleotide base “C” may be assigned a value 01 (base-2), the DNA nucleotide base “T” may be assigned a value 10 and the DNA nucleotide base “G” may be assigned a value 11. These binary-to-quaternary mappings, and their complements, are used in the examples provided herein, but it should be understood that any similar mapping may be used.

In one example, the encoding system 105 performs a “direct encoding” process when preparing input data for memorialization in DNA. Direct encoding includes the binary-to-quaternary mappings to translate or convert each pair of bits into a single nucleotide. For example, input data of 010010110100 may be directly encoded as a DNA sequence or DNA strand of CATGCA. This data, when embodied in DNA form, may be referred to herein as “DNA data.” Such direct encoding of data yields one nucleotide of DNA data for every two bits of input data. Other more complex encoding processes are described herein.

The data storage system 100 may also include a synthesis system 110. In an example, the synthesis system 110 writes or otherwise manufactures DNA strands based on the data provided by the encoding system 105. For example, using a series of chemical steps or processes, the synthesis system 110 creates and assembles the various DNA bases (e.g., the ACTG bases) are assembled to mirror the base-4 representation determined from the encoding process. Although chemical steps or processes are mentioned, the synthesis system 110 may use other synthesis techniques, and the synthesis system 110 includes both hardware components configured to create such DNA strands as well as software and/or electronic components for controlling those hardware components.

Continuing with the example above, during synthesis, since the digital data of 010010110100 is represented as CATGCA, the synthesis system 110 would first generate and/or identify a “C” base. An “A” base would then be generated and/or identified and be attached to the “C” base. A “T” base would then be generated and/or identified and be attached to the “CA” combination that was previously generated. This process repeats until the entire DNA strand (e.g., CATGCA) is created. The terms “created”, “generated”, or “synthesized”, and their variants, may be used interchangeably herein when referring to the making of a real-world synthetic string of DNA. Further, the terms “DNA strand” and “DNA sequence” may also be used interchangeably to refer to the synthetic DNA molecule created during the processes described herein, or to a mathematical representation of that DNA strand, depending on context.

As previously discussed, indel errors may occur during synthesis, storage and/or sequencing. However, indel errors occur less frequently at the beginning of the DNA strand and increase in frequency toward the end of the DNA strand.

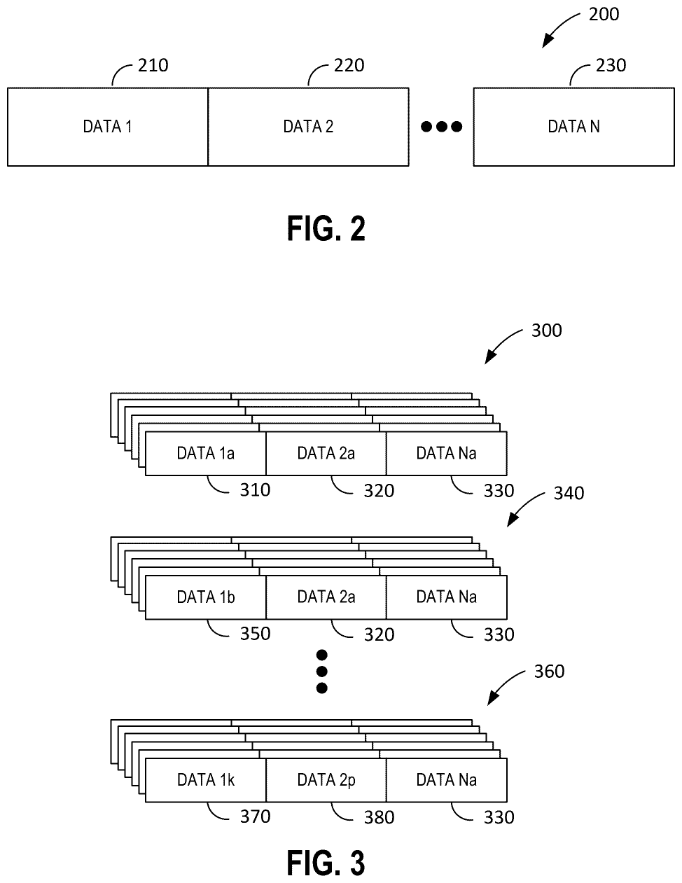

Based on the fact that indel errors occur less frequently at the beginning of the DNA strand when compared to the end, the encoding system 105 and/or the synthesis system 110 divides the DNA strand into a series of N reliability tiers. For example and referring to FIG. 2, FIG. 2 illustrates a DNA strand 200 having N reliability tiers. In an example, each tier is associated with a particular type of data.

For example, the first tier 210 of the DNA strand stores a first type of data (e.g., Data 1), the second tier 220 of the DNA strand 200 stores a second type of data (e.g., Data 2) and the Nth tier 230 of the DNA strand 200 stores an Nth type of data (e.g., Data N). In an example, the data has a “type” based, at least in part, on identified level of importance, a performance level (e.g., different tiers will have longer/shorter reading times based, at least in part, on how far the tier is within the DNA strand 200) a determined functionality and/or a determined/identified criticality.

For example, data that is used for storage management (e.g., data corresponding to headers, data indexing, address information, a strand identifier) and/or data that is used for a decoding process (e.g., data statistics, data associated with cyclic redundancy checks) may be identified or defined as important or critical data. As such, this type of data is stored in the first tier 210 of the DNA strand 200.

The second tier 220 of the DNA strand 200 stores data that that is less critical when compared with the data stored in first tier 210. Likewise, the Nth tier 230 stores data that is less important/critical when compared with the data that is stored in the first tier 210 through the N−1 tier. For example, the data that is stored in the Nth tier may include data that is “nice to have” but is not identified as critical. In an example, the data that is “nice to have” includes, but is not limited to, data logs, additional parity information (e.g., parity information that is in addition to a minimum amount of parity information), additional cyclic redundancy check data and/or pointer data (e.g., information that points to the next DNA strand).

Each tier may include any number of nucleotides. In an example, a size or length of each tier may be uniform. For example, and referring back to the example described with respect to FIG. 1, when the encoding system 105 and/or the sequencing system 110 generates the DNA strand CATGCA, the first tier 210 may include the first “C” base, the second tier 220 may include the first “A” base, the third tier may include the “T” base and so on until the Nth 230 tier would include the last “A” base.

In another example, the first tier 210 may include the nucleotides “CA”, the second tier 220 may include the nucleotides “TG” and the Nth tier may include the second nucleotides “CA”. Although specific lengths and nucleotides (and combinations of nucleotides) are mentioned, these are for example purposes only and each tier may include M DNA nucleotides.

In another example, the size and/or length of each tier may be different. For example, the first tier 210 of the DNA strand 200 may include P DNA nucleotides while a second tier 220 of the DNA strand 200 may include Q DNA nucleotides.

Because indel errors occur less frequently at the beginning of the DNA strand 200 and more frequently toward the end of the DNA strand, an amount of time required for a decoding process and/or an error correction process may vary across the tiers. For example, a decoding and/or error correction process for the first tier 210 may take a first amount of time, the decoding and/or error correction process for the second tier 220 may take a second amount of time and the decoding and/or error correction process for the Nth tier 230 may take a third amount of time.

In another example, because data at the beginning of the DNA strand 200 is less susceptible to errors, fewer copies of data associated with the first tier 210 may need to be encoded, synthesized and/or stored when compared with data associated with later tiers.

FIG. 3 illustrates multiple DNA strands that have the same data in later tiers but different data in the beginning tiers. For example, a first DNA strand 300 may include N tiers and each tier may store or otherwise be associated with data. In the example shown, the first DNA strand 300 includes a first tier 310, a second tier 320 and a Nth tier 330. The first tier 310 stores Data 1a, the second tier 320 stores Data 2a and the Nth tier 330 stores Data Na.

In an example, any number of copies of the first DNA strand 300 may be generated during an encoding and/or decoding process and stored by a DNA-based data storage device. In the example shown, six copies of the first DNA strand 300 are created.

However, because data near the beginning of the DNA strand is less susceptible to errors when compared with data at the end of the DNA strand, a second DNA strand 340 may have different data in the earlier tiers but the same data in the later tiers. For example, the second DNA strand 340 includes a first tier 350 that stores Data 1b. In an example, Data 1a and Data 1b may be a first type of data (e.g., data having the same or similar level of importance and/or functionality). However, the second tier 320 of the second DNA strand 340 stores Data 2a and the Nth tier 330 of the second DNA strand 340 stores Data Na. Thus, up to this point in the example, the DNA-based data storage device has stored six copies Data 1a, six copies of Data 1b, twelve copies of Data 2a and twelve copies of data Na.

Additionally, a Nth DNA strand 360 stores Data 1k in a first tier 370, stores Data 2p in a second tier 380 and stores Data Na in a Nth tier 330. Thus, in this example, the DNA-based data storage device has stored six copies Data 1a, six copies of Data 1b, six copies of Data 1k, twelve copies of Data 2a, P copies of Data 2p and N copies of data Na.

Referring back to FIG. 1, when the synthesis process is complete, the DNA strand is stored in a physical storage medium such as, for example, a dense storage system 135 (e.g., one or more synthetic DNA molecules). The dense storage system 135 enables the synthesized DNA strand to be stored and subsequently accessed. In an example, any storage medium capable of storing DNA-based data may be used as the dense storage system 135.

Once the DNA strand has been stored, it may be subsequently accessed and prepared for sequencing (e.g., being read). As part of the preparation process, multiple copies of the DNA strand may be generated. In an example, an amplification system 115 of the data storage system 100 may ensure that multiple copies of the DNA data are generated. In an example, the amplification system 115 may be used to create the multiple copies of each DNA strand shown and described with respect to FIG. 3. For example, the amplification system 115 may be responsible for creating the copies of the first DNA strand 300, the second DNA strand 340 and/or the Nth DNA strand 360.

A sequencing system 120 may then be used to read DNA strands from the dense storage system 135. In an example, the sequencing system 120 determines and/or identifies an order of the DNA symbols (e.g., ACTG) in a DNA segment of a DNA strand that is being read. The sequencing system 120 may use a variety of sequencing methods such as, for example, sequencing by synthesis, nanopore sequencing, and the like.

Once the DNA strand has been read, a decoding system 125 maps the DNA symbols (e.g., in base-4) back to digital data (e.g., in base-2). For example, in “direct decoding,” if the decoding system 125 receives CATGCA as the DNA strand, the decoding process performed by the decoding system 125 would return 010010110100 (e.g., using the corollary of the binary-to-quaternary mappings discussed above) to a requesting computing device (e.g., computing device 150). Other more complex decoding processes are described herein. In some examples, the inverse of the encoding process used to make the DNA strand may be used as the decoding process.

In some examples, errors may occur during the synthesis process, the storage process and/or the sequencing process. These errors may be, for example, insertion and deletion (“indel”) errors and/or substitution errors. For example, during a synthesis process in which the DNA strand CATGCA is being synthesized, one or more symbols may be deleted or lost during the creation of the DNA molecule. As a result, a DNA strand CTGCA may be stored by the dense storage system 135. In another example, during a synthesis process in which the DNA strand CATGCA is being synthesized, an additional symbol may be added. As a result, a DNA strand CCATGCA may be stored by the dense storage system 135. Although a single insertion error and a single deletion error are discussed, multiple deletions and/or insertions may occur in a synthesis process. Additionally, these errors may occur during storage and/or during a sequencing process (e.g., during the writing/creating of the DNA molecule, or during the reading/sequencing of the DNA molecule).

In yet another example, during a synthesis process in which the DNA strand CATGCA is being synthesized, the synthesis system 110 may substitute one symbol for another. As a result, a DNA strand TATGCA may be stored in the dense storage system 135 instead of the DNA strand CATGCA. In an example, multiple substitution errors (along with one or more indel errors) may occur during the synthesis process, during storage and/or during a sequencing process.

In order to address the above, the data storage system 100 may include an error correction system 130. The error correction system 130 may be part of the decoding system 125. The error correction system 130 may use various processes to detect and address indel errors and/or substitution errors. In one example, indel errors may be addressed by generating multiple copies of a particular DNA strand or multiple copies of different tiers of the particular DNA strand. For example, the error correction system 130 may be used to generate one or more of the first DNA strand 300 (FIG. 3) and/or one or more of its respective tiers, the second DNA strand 340 (FIG. 3) and/or one or more of its respective tiers and/or the Nth DNA strand 360 (FIG. 3) and/or one or more of its respective tiers.

In an example, once the copies of the DNA strand are generated, each of the copies are read and compared to generate a consensus codeword. For example, a first DNA symbol (or DNA segment or DNA tier consisting of multiple DNA symbols) of a first DNA strand is compared with a first DNA symbol (or DNA segment or DNA tier consisting of multiple DNA symbols) from one or more of the copies of the DNA strand. This process repeats for each DNA symbol (or DNA segment) in the DNA strand.

The error correction system 130 may then determine, based on consensus data across all of the copies, which DNA symbol is the correct (or most correct) DNA symbol for that particular index (or DNA segment). For example, the most prominent DNA symbol in each index of the DNA strand may be selected as the correct DNA symbol and a consensus codeword is generated for each DNA segment. The resulting consensus codeword is mapped to corresponding ones and zeros and is provided to the decoding system 125 (e.g., a low-density parity check (LDPC) decoder).

In an example, the consensus data generated by the error correction system 130 may be referred to herein as hard bit data or hard information. The error correction system 130 and/or the decoding system 125 described in the present application may also generate and use soft-bit data or soft information using information associated with the consensus data (or using information that is obtained while the consensus data is determined).

For example, once multiple copies of a particular DNA strand have been copied or otherwise generated (e.g., by the amplification system 115), each copy of the DNA strand that is associated with a received codeword (e.g., DNA-based data that is to be read from the dense storage system 135), is divided into k DNA segments (where k is equal to or greater than two). Each DNA segment has a DNA segment length n (where n is equal to or greater than one).

For example, the DNA strand CATGCA may be divided into two different DNA segments having a length of three. In an example, the segments may be associated with the reliability tiers that were previously discussed.

For example, a first DNA segment may be “CAT” and a second DNA segment may be “GCA”. In another example, the DNA strand CATGCA may be divided into three different DNA segments having a length of two. In this example, the first DNA segment would be “CA”, the second DNA segment would be “TG” and the third DNA segment would be “CA”. In yet another example, the DNA strand CATGCA may be divided into six different DNA segments having a length of one. In this example, the first DNA segment would be “C”, the second DNA segment would be “A”, the third DNA segment would be “T”, the fourth DNA segment would be “G” and so on.

The data storage system 100 may also include a dense storage management system 140. In an example, the dense storage management system 140 controls the various operations and/or processes that are carried out by and/or on the dense storage system 135. The operations and/or processes may include the mechanics of storage and retrieval of the DNA data and/or information storage management (e.g., making copies of data, deleting data, selecting subsets of the data, etc.).

The data storage system 100 may also include a control system 145. The control system 145 may include at least one processor, at least one controller and/or other such control circuitry. The control system 145 may include circuitry for executing instructions from the computing device 150 (or from another source) and/or providing instructions to the various subsystems of the data storage system 100.

This process of converting the input data (base-2) into DNA molecule(s) (base-4) and subsequently converting the DNA molecule(s) (base-4) into output data (base-2), including any of the interim processes that the data and/or DNA molecules undergo, may be referred to herein as “the DNA storage channel.” It should be understood that, in these examples, it is an objective of the DNA storage channel and the systems and methods described herein to generate output data that is as close to identical to the input data as possible.

In an example, the DNA-based storage system may use parity information to correct substation errors and/or indel errors that occur or are otherwise detected as part of the sequencing, storage and/or synthesis processes of a DNA strand. Additionally, the amount of parity information available to each segment or portion of the DNA strand may differ. For example, a first amount of parity information is available to a first segment of the DNA strand that is at or near the beginning of the DNA strand while a second amount of parity information is available to a segment of the DNA strand that is at or near the end of the DNA strand.

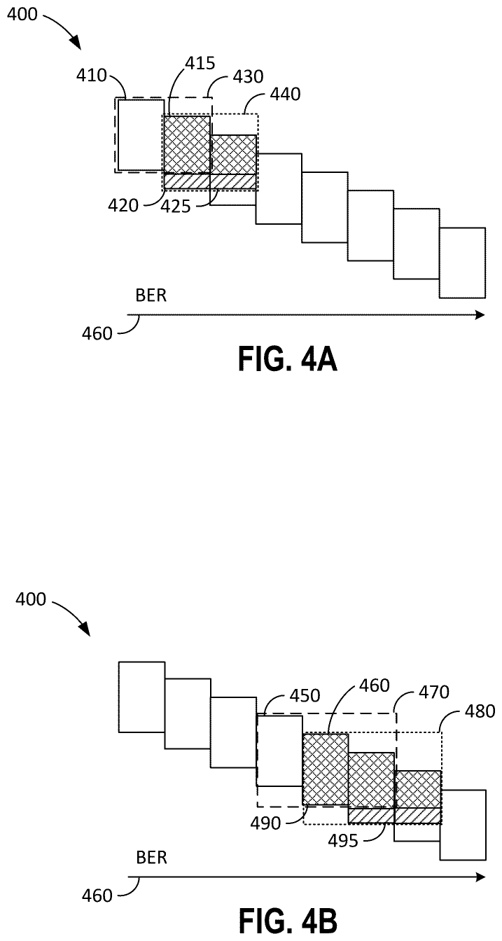

FIG. 4A illustrates how a first amount of parity information is provided to a first portion of a DNA 400 strand during an error correction process according to an example. In an example, the error correction process and/or the allocation of parity information to different portions of the DNA strand 400 may be performed by the encoding system 105 of the data storage device 100, the decoding system 125 of the data storage device 100 and/or the error correction system 130 of the data storage device 100 shown and described with respect to FIG. 1.

As previously discussed, the beginning of the DNA strand 400 is less susceptible to errors when compared with the end of the DNA strand 400. This is represented by the arrow 460. Because the beginning of the DNA strand 400 is less susceptible to errors when compared with the other portions of the DNA strand 400, an error correction process may implement the use spatially coupled LDPC codes and associated parity information to correct any errors that are detected during an encoding and/or a decoding process.

In an example, the amount of parity information that is provided to the error correction process increases as the bit error rate (BER) increases. Additionally, the spatially coupled LDPC codes are interconnected in a spatial manner such that a chain-like structure is created. The coupling includes connecting adjacent LDPC codes in a specific pattern.

In an example and as shown in FIG. 4A, the DNA strand 400 is separated into portions or segments (e.g., represented by the rectangular boxes). For example, the DNA strand 400 may include a first portion 410 and a second portion 420. Although two portions are identified, the DNA strand 400 may include any number of portions. In an example, the first portion 410 and/or the second portion 420 are equivalent to reliability tiers such as previously described.

During a first decoding and/or error correction process, a first tile 430 sets the metes and bounds of the portion (or portions) of the DNA strand 400 that will be decoded. In an example, some parity information is provided to the decoding and/or error correction process. However, because the first portion contains few errors, a first amount of parity information may be used. When the first decoding and/or error correction process is complete, a second tile 440 defines the metes and bounds of a second decoding and/or error correction process. In an example, a size of the second tile 440 may be equivalent or substantially equivalent to a size of the first tile 430.

In an example, the second tile 440 overlaps at least a portion of the first tile 410. The overlapping portion is illustrated by a first fill pattern 415. The overlapping portion was already decoded and/or error corrected during the first (or a previous) decoding and/or error correction process. Thus, during the second decoding and/or error correction process, the only portion of the DNA strand 400 that needs to be decoded (at least with respect to the second tile 440) is the non-overlapping portion (e.g., the portion illustrated by a second fill pattern 425).

In an example, because the overlapping portion was already successfully decoded and/or error corrected, the overlapping portion is already error free. As such, the overall tile BER is lower than a standalone decoding of the same tile. Thus, as additional tiles are added and/or as the tile is moved along different portions of the DNA strand 400, an amount of parity information that is provided to the decoding and/or error correction process increases. As the amount of parity information increases, so does the error correction capability of the error correction system.

FIG. 4B illustrates how a second overlapping portion is provided to a second portion of the DNA strand 400 of FIG. 4A according to an example. In this example, the decoding and/or error correction process has continued to a P portion 450 of the DNA strand 400 and a Q portion 460 of the DNA strand 400. During a P decoding and/or error correction process, a P tile 470 sets the metes and bounds of the portion (or portions) of the DNA strand 400 that will be decoded. In an example and as previously discussed, information from a P−1 decoding and/or error correction process was provided to the error correction system and/or the decoding system. When the P decoding and/or error correction process is complete, a Q tile 480 defines the metes and bounds of a Q decoding and/or error correction process. In an example, a size of the P tile 470 and a size of the Q tile 480 may be larger when compared to a size of the first tile 430 and/or the size of the second tile 440. As such, more information may be included in the decoding and/or error correction processes. In another example, the size of the P tile 470 and/or a size of the Q tile 480 may be equivalent or substantially equivalent to the size of the first tile 430.

Like the previous example, the Q tile 480 overlaps at least a portion of the P tile 470. The overlapping portion is illustrated by a first fill pattern 490. As previously discussed, the overlapping portion was already decoded and/or error corrected. Thus, during the Q decoding and/or error correction process, the only portion of the DNA strand 400 that needs to be decoded and/or corrected (at least with respect to the Q tile 480) is the non-overlapping portion (e.g., the portion illustrated by a second fill pattern 495). Because the overlapping portion was already successfully decoded and/or error corrected, the overlapping portion may be used as parity information during the Q decoding and/or error correction process.

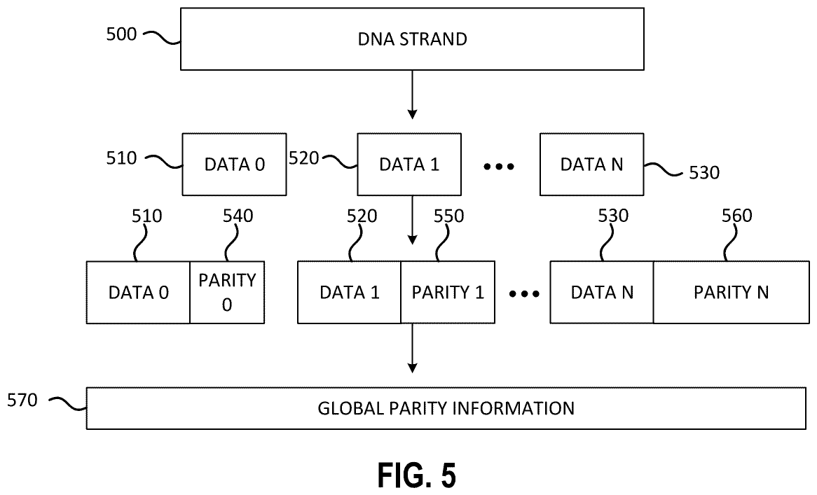

FIG. 5 illustrates a DNA strand 500 being divided into one or more portions or sub-codes, with each portion of sub-code being associated with different amounts of parity information according to an example. In an example, the DNA strand 500 is divided into sub-codes and/or is associated with different amounts of parity information as part of an encoding process, as part of a decoding process and/or as part of an error correction process performed by a data storage device, such as, for example the data storage device 100 shown and described with respect to FIG. 1.

Thus, one more of the operations described below may be implemented by an encoding system 105, a decoding system 125 and/or an error correction system 130. In an example, the amount of parity information that is provided to each sub-code may be based, at least in part, on the properties of the DNA storage channel in which a beginning portion of the DNA strand 500 is less susceptible to errors when compared with other portions of the DNA strand 500.

In an example, the data storage system implements a sub-code architecture during encoding, decoding and/or error correction. As part of the sub-code architecture, the DNA strand 500 is divided or segmented into two or more short DNA strands. For example, the DNA strand 500 is divided into N short DNA strands represented as Data 0 510, Data 1 520 and Data N 530.

Additionally, each short DNA strand is associated with parity information. In an example, the parity information is associated with each short DNA strand during an encoding process, a decoding process and/or an error correction process. In an example, each short DNA strand is associated with its own unique parity information. For example, Data 0 510 is associated with Parity 0 540, Data 1 520 is associated with Parity 1 550 and Data N 530 is associated with Parity N 560.

However, because the beginning of the DNA strand 500 has fewer errors when compared with later portions of the DNA strand 500, each short DNA strand may be associated with different amounts of parity information. For example, the Parity 0 540 may include a first amount of parity information, Parity 1 550 may include a second amount of parity information and Parity N 560 may include a third amount of parity information such that Parity 0 540<Parity 1 550<Parity N 560.

In an example, the combination of each short DNA strand (or the data associated with each short DNA strand) and its associated parity information is referred to as a sub-code. Additionally, each short DNA strand may be encoded, decoded and/or error corrected separately from the other short DNA strands. For example, Data 0 220 may be encoded/decoded separately from Data 1 230. The parity information associated with each short DNA strand is used to correct any errors that may occur during an encoding/synthesis process and/or a decoding/sequencing process.

In addition to the parity information that is associated with each short DNA strand, global parity information 570 may also be used to correct any errors that are detected during the encoding/decoding processes. In an example, the global parity information 570 includes data associated with some or all of the short DNA strands, along with the parity information associated with each short DNA strand.

In an example, the global parity information 570 includes information from short DNA strands and/or its associated parity information that have already been decoded and/or error corrected. For example, the global parity information 570 includes data associated with Data 0 510, Data 1 520 and/or Data N 530 and may also include Parity 0 540, Parity 1 550 and/or Parity N 560.

In another example, parity information is added to the global parity information 570 incrementally. For example, the global parity information 570 includes Data 0 510 and/or Parity 0 540 when Data 0 510 has been successfully decoded and/or error corrected. When Data 1 520 has been successfully decoded and/or error corrected, the Data 1 520 and/or Parity 1 550 may be included or otherwise added to the global parity information 570. Because the global parity information 570 includes some or all of the data associated with each short DNA strand and the parity information associated with each short DNA strand, the overall correction capabilities of the data storage device is improved.

In an example, when various sub-codes, tiers and/or tiles are decoded and/or error corrected, a data storage device (e.g., the data storage device 100 (FIG. 1)) can use extracted information on data patterns and error patterns associated with each DNA strand. This extracted information may be used and/or analyzed by a decoding system and/or an error correction system to further improve its correction capabilities.

For example, the extracted information may include a determination of a probability of errors per written symbol and/or a probability that one symbol will be substituted for an additional symbol. In another example, the extracted information may include information regarding the probability of an error occurring in a repeating symbol as a function of the number of repetitions of that symbol.

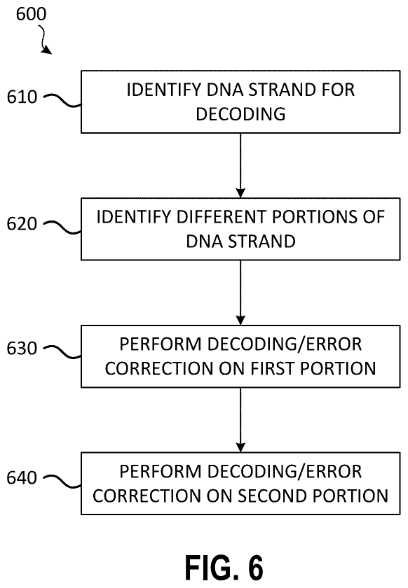

FIG. 6 illustrates a method 600 for decoding a DNA strand according to an example. In an example, the method 600 is performed by one or more systems of a DNA-based storage system such as, for example, the data storage device 100 shown and described with respect to FIG. 1.

In an example, the method 600 begins when a DNA strand is identified (610) for decoding. In an example, when the DNA strand is identified for decoding, multiple copies of the DNA strand are generated. In an example, an amplification system of the data storage system ensures that multiple copies of the DNA data are generated. In an example, the amplification system 115 creates the multiple copies of each DNA strand shown and described with respect to FIG. 3. For example, the amplification system creates the copies of the first DNA strand 300, the second DNA strand 340 and/or the Nth DNA strand 360.

When the DNA strand is identified, one or more different portions of the DNA strand are identified (620). For example, the DNA strand may be portioned or divided based, at least in part, on one or more determined reliability tiers. For example, a first portion of the DNA strand may define a first reliability tier, a second portion of the DNA strand may define a second reliability tier and so on.

A sequencing system may then be used to read one or more portions of the DNA strand from a dense storage system. Once the DNA strand has been read, a decoding system maps the DNA symbols back to digital data such as previously described. For example, the decoding system may perform (630) a first decoding/error correction process on a first portion of the DNA strand. In such an example and because the first portion of the DNA strand includes fewer errors than other portions of the DNA strand, a first amount of parity information may be used to decode and/or error correct the first portion.

When the first portion has been decoded and/or error corrected, the decoding system performs (640) a second decoding/error correction process on a second portion of the DNA strand. In such an example and because the second portion of the DNA strand may include more errors when compared with the first portion, but fewer errors than other portions of the DNA strand, a second amount of parity information may be used to decode and/or error correct the second portion. This process may continue N times for each segment or portion of the DNA strand.

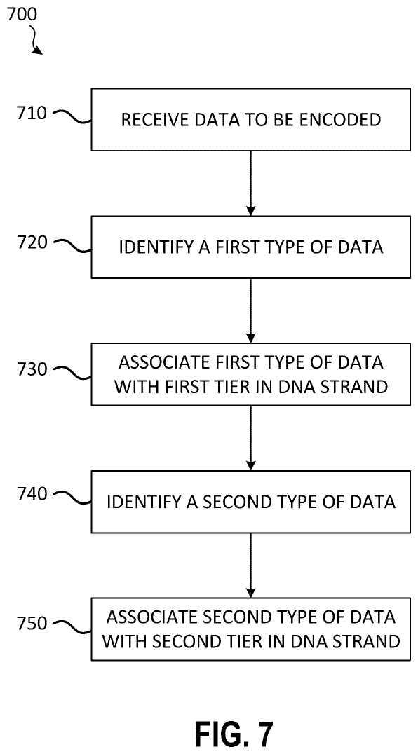

FIG. 7 illustrates a method 700 for encoding data for a DNA strand having different reliability tiers according to an example. In an example, the method 700 is performed by one or more systems of a DNA-based storage system such as, for example, the data storage device 100 shown and described with respect to FIG. 1. For example, the method 700 may be performed by an encoding system 105 and/or a synthesis system 110 of the data storage device 100.

In an example, the method 700 begins when data to be encoded on a DNA strand is received (710). For example, the encoding system receives digital/binary information and/or data from a source. When the data is received, the encoding system converts or maps the ones and zeros of the original data into one or more DNA strands using the synthetic DNA nucleotide bases A, C, T, and G such as previously described.

Additionally, when the data is received, the encoding system may also determine or identify (720) a type of the data. For example, the encoding system determines a functionality that the data will perform and/or an importance level of the received data. In an example, the importance level may be associated with an identifier that is included as part of the received data.

The encoding system and/or a synthesis system of the data storage system writes or otherwise manufactures DNA strands based on the received data. For example, during a synthesis process, the various DNA bases that comprises the first type of data are assembled or otherwise associated (730) with a first tier of the DNA strand. The above processes may be repeated for other types of data.

For example, the encoding system may also determine or identify (740) a second type of data that is included in the originally received data. For example, the encoding system determines a functionality that the second type of data will perform and/or an importance level of the second type of data.

When the second type of data is identified, the encoding system and/or the synthesis system assembles or associates (750) the various DNA bases that comprises the second type of data with a second tier of the DNA strand.

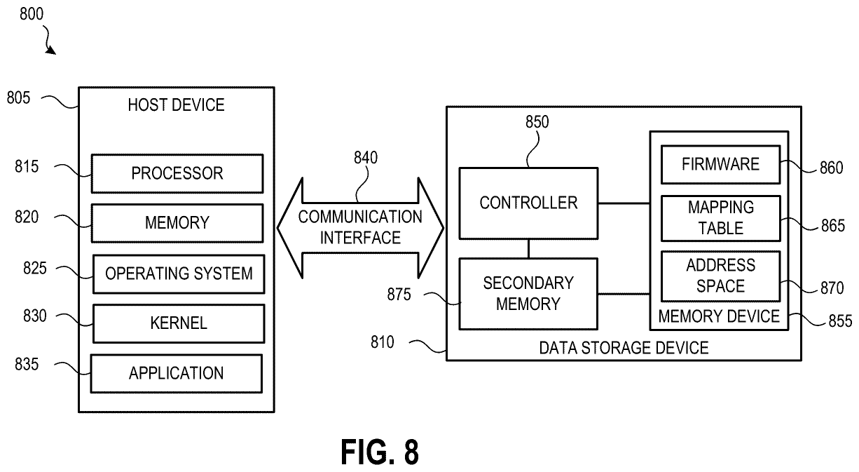

FIG. 8 is a block diagram of a system 800 that includes a host device 805 and a data storage device 810 according to an example. In an example, the host device 805 may be similar to the computing device 150 shown and described with respect to FIG. 1, and may be used to perform any or all of the operations described herein. The host device 805 includes at least one processor 815 and a memory device 820 (e.g., main memory). The memory device 820 may include an operating system 825, a kernel 830 and/or an application 835.

The at least one processor 815 can execute various instructions, such as, for example, instructions from the operating system 825 and/or the application 835. The at least one processor 815 may include circuitry such as a microcontroller, a Digital Signal Processor (DSP), an Application-Specific Integrated Circuit (ASIC), a Field Programmable Gate Array (FPGA), hard-wired logic, analog circuitry and/or various combinations thereof. In an example, the at least one processor 815 includes a System on a Chip (SoC). In examples in which two or more processors are used, each processor may perform various functionalities as described herein. For example, a first processor performs a first set of features/functionality while a second processor performs a second set of features/functionality.

In an example, the memory device 820 can be used by the host device 805 to store data used by the at least one processor 815. Data stored in the memory device 820 may include instructions provided by the data storage device 810 via a communication interface 840. The data stored in the memory device 820 may also include data used to execute instructions from the operating system 825 and/or one or more applications 835. In an example, the memory 820 is volatile memory, such as, for example, Dynamic Random Access Memory (DRAM).

In an example, the operating system 825 may create a virtual address space for the application 835 and/or other processes executed by the at least one processor 815. The virtual address space may map to locations in the memory device 820. The operating system 825 may include or otherwise be associated with a kernel 830. The kernel 830 may include instructions for managing various resources of the host device 805 (e.g., memory allocation), handling read and write requests and so on.

The communication interface 840 communicatively couples the host device 805 and the data storage device 810. The communication interface 840 may be a Serial Advanced Technology Attachment (SATA), a PCI express (PCIe) bus, a Small Computer System Interface (SCSI), a Serial Attached SCSI (SAS), Ethernet, Fibre Channel, or WiFi. As such, the host device 805 and the data storage device 810 need not be physically co-located and may communicate over a network such as a Local Area Network (LAN) or a Wide Area Network (WAN), such as the internet. In addition, the host device 805 may interface with the data storage device 810 using a logical interface specification such as Non-Volatile Memory express (NVMe) or Advanced Host Controller Interface (AHCI).

The data storage device 810 includes at least one controller 850 and a memory device 855 (e.g., volatile and/or non-volatile memory). The memory device 855 (and/or portions of the memory device 855) may also be referred to as a storage medium. The memory device 855 includes a number of storage elements. In an example, each storage element is a chip or a memory die that is used to store data.

For example, the memory device 855 may include a first memory die and a second memory die. In an example, the first memory die and the second memory die include non-volatile memory elements such as, for example, NAND flash memory elements and/or NOR flash memory elements. Although two memory dies are mentioned, the memory device 855 may include any number of storage elements. For example, the storage elements may take the form of solid-state memory such as, for example, 2D NAND, 3D NAND memory, multi-level cell memory, triple level cell memory, quad-level cell memory, penta-level cell memory or any combination thereof.

The at least one controller 850 may include circuitry for executing instructions. The instructions may originate from firmware 860 associated with the data storage device 810. In another example, the instructions may originate from the host device 805. Accordingly, the at least one controller 850 may include circuitry such as one or more processors, a microcontroller, a DSP, an ASIC, an FPGA, hard-wired logic, analog circuitry and/or a combination thereof. In another example, the controller 850 may include a SoC.

The data storage device 810 may also include secondary memory 875. The secondary memory 875 may be a rotating magnetic disk or non-volatile solid-state memory, such as flash memory. While the description herein refers to solid-state memory generally, it is understood that solid-state memory may comprise one or more of various types of memory devices such as flash integrated circuits, NAND memory (e.g., single-level cell (SLC) memory, multi-level cell (MLC) memory (i.e., two or more levels), or any combination thereof), NOR memory, EEPROM, other discrete Non-Volatile Memory (NVM) chips, or any combination thereof.

In some examples, the memory device 855 is capable of storing data at a byte-addressable level, as opposed to other types of non-volatile memory that have a smallest writable data size such as a page size of 4 KB or a sector size of 512 Bytes.

In some examples, the memory device 855 may also store a mapping table 865 and/or an address space 870. In some examples, the controller 850 can associate portions of data stored in the secondary memory 875 with unique identifiers. The unique identifiers may be stored in the memory device 855 and be used by the operating system 825 to access stored data. For example, the mapping table 865 can provide a mapping of unique identifiers with indications of physical locations (e.g., Physical Block Addresses (PBAs)) where the corresponding portions of data are stored in the memory device 855 and/or the secondary memory 875.

In some examples, the firmware 860 may store, maintain, be associated with or otherwise have access to a mapping table (e.g., mapping table 865) that stores and/or maintains mapping information for the various DNA strands such as described above.

As briefly discussed above, the memory device 855 may also include address space 870. The address space 870 can serve as at least a portion of an address space used by the at least one processor 815. In an example, the address space 870 can store data at a byte-addressable level that can be accessed by the at least one processor 815 (e.g., via the communication interface 840).

For example, the data storage device 810 may provide the host device 805 with an indication of the address space 870. The host device 805 may then associate an address range for the address space 870 and an indication that this address range is to be used as a byte-addressable address space, such as for a page cache.

In another example, the host device 805 may manage the data storage device 810 such that the at least one processor 815 can directly access address space 870. For example, the data storage device 810 may provide logical to physical address translation information to the host device 805, which can be called by the host device 805 and executed by the at least one processor 815 and/or the at least one controller 850. In some examples, the at least one controller 850 may include or otherwise be associated with a flash translation layer (FTL). The FTL may map the logical block addresses to the physical addresses of the memory device 855.

Although FIG. 8 illustrates the host device 805 being separate from the data storage device 810, the host device 805 and the data storage device 810, as well the various components described, may be part of a single device or part of multiple devices.

The term computer-readable media as used herein may include computer storage media. Computer storage media may include volatile and nonvolatile, removable and non-removable media implemented in any method or technology for storage of information, such as computer readable instructions, data structures, or program modules. Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by a computing device. Any such computer storage media may be part of the computing device. Computer storage media does not include a carrier wave or other propagated or modulated data signal.

Additionally, examples described herein may be discussed in the general context of computer-executable instructions residing on some form of computer-readable storage medium, such as program modules, executed by one or more computers or other devices. By way of example, and not limitation, computer-readable storage media may comprise non-transitory computer storage media and communication media. Generally, program modules include routines, programs, objects, components, data structures, etc., that perform particular tasks or implement particular abstract data types. The functionality of the program modules may be combined or distributed as desired in various examples.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Based on the above, examples of the present disclosure describe a DNA-based storage system, comprising: a DNA strand comprising a first reliability tier and a second reliability tier, wherein: the first reliability tier is associated with a first type of data; and the second reliability tier is associated with a second type of data. In an example, the first reliability tier is associated with a first bit error rate (BER) and the second reliability tier is associated with a second BER. In an example, the first reliability tier is associated with a first amount of parity information and the second reliability tier is associated with a second amount of parity information. In an example, the DNA-based storage system also includes multiple copies of the DNA strand wherein: a first copy of the DNA strand includes a first portion associated with first data and a second portion associated with second data; and a second copy of the DNA strand includes a first portion associated with third data and a second portion associated with the second data. In an example, the first type of data includes data relating to storage management. In an example, the first type of data includes data relating to a decoding process. In an example, the first type of data has a higher level of importance when compared to the second type of data.

The present disclosure also describes a DNA-based storage system, comprising: an encoding system operable to: receive data to be encoded on a DNA strand; identify a first type of data in the received data; identify a second type of data in the received data; associate the first type of data with a first tier in the DNA strand; and associate the second type of data with a second tier in the DNA strand. In an example, the encoding system is further operable to: associate a first amount of parity information with the first tier; and associate a second amount of parity information with the second tier. In an example, the second amount of parity information includes global parity information. In an example, the DNA-based storage system also includes a decoding system, wherein the decoding system is operable to: generate multiple copies of the DNA strand wherein: a first copy of the DNA strand includes a first portion associated with the first type data and a second portion associated with the second type of data; and a second copy of the DNA strand includes a first portion associated with a third type of data and a second portion associated with the second type of data. In an example, the first type of data has a higher level of importance when compared to the second type of data. In an example, the first tier has a first bit error rate (BER) and the second tier has a second, higher BER.

Other examples describe a DNA-based storage system, comprising: means for encoding data on a DNA strand, the DNA strand comprising: a first reliability tier that is associated with a first type of data; and a second reliability tier that is associated with a second type of data; means for storing the DNA strand; and means for decoding the DNA strand. In an example, the means for encoding data on the DNA strand generates a first amount of parity information for the first reliability tier and generates a second amount of parity information for the second reliability tier. In an example, the means for decoding utilizes the first amount of parity information when decoding data associated with the first reliability tier and uses the second amount of parity information when decoding data associated with the second reliability tier. In an example, at least one of the first amount of parity information and the second amount of parity information includes global parity information. In an example, the means for storing the DNA strand stores multiple copies of the DNA strand and wherein: a first copy of the DNA strand includes a first portion associated with first data and a second portion associated with second data; and a second copy of the DNA strand includes a first portion associated with third data and a second portion associated with the second data. In an example, the first data and the third data are the first type of data. In an example, the first type of data has a higher level of importance when compared to the second type of data.

The description and illustration of one or more aspects provided in the present disclosure are not intended to limit or restrict the scope of the disclosure in any way. The aspects, examples, and details provided in this disclosure are considered sufficient to convey possession and enable others to make and use the best mode of claimed disclosure.

The claimed disclosure should not be construed as being limited to any aspect, example, or detail provided in this disclosure. Regardless of whether shown and described in combination or separately, the various features (both structural and methodological) are intended to be selectively rearranged, included or omitted to produce an example with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

Aspects of the present disclosure have been described above with reference to schematic flowchart diagrams and/or schematic block diagrams of methods, apparatuses, systems, and computer program products according to examples of the disclosure. It will be understood that each block of the schematic flowchart diagrams and/or schematic block diagrams, and combinations of blocks in the schematic flowchart diagrams and/or schematic block diagrams, can be implemented by computer program instructions. These computer program instructions may be provided to a processor of a computer or other programmable data processing apparatus to produce a machine, such that the instructions, which execute by way of the processor or other programmable data processing apparatus, create means for implementing the functions and/or acts specified in the schematic flowchart diagrams and/or schematic block diagrams block or blocks.

References to an element herein using a designation such as “first,” “second,” and so forth does not generally limit the quantity or order of those elements. Rather, these designations may be used as a method of distinguishing between two or more elements or instances of an element. Thus, reference to first and second elements does not mean that only two elements may be used or that the first element precedes the second element. Additionally, unless otherwise stated, a set of elements may include one or more elements.

Terminology in the form of “at least one of A, B, or C” or “A, B, C, or any combination thereof” used in the description or the claims means “A or B or C or any combination of these elements.” For example, this terminology may include A, or B, or C, or A and B, or A and C, or A and B and C, or 2A, or 2B, or 2C, or 2A and B, and so on. As an additional example, “at least one of: A, B, or C” is intended to cover A, B, C, A-B, A-C, B-C, and A-B-C, as well as multiples of the same members. Likewise, “at least one of: A, B, and C” is intended to cover A, B, C, A-B, A-C, B-C, and A-B-C, as well as multiples of the same members.

Similarly, as used herein, a phrase referring to a list of items linked with “and/or” refers to any combination of the items. As an example, “A and/or B” is intended to cover A alone, B alone, or A and B together. As another example, “A, B and/or C” is intended to cover A alone, B alone, C alone, A and B together, A and C together, B and C together, or A, B, and C together.

What is claimed is:

1. A DNA-based storage system, comprising:
a DNA strand comprising a first reliability tier and a second reliability tier, wherein:
the first reliability tier is associated with a first type of data; and
the second reliability tier is associated with a second type of data.

2. The DNA-based storage system of claim 1, wherein the first reliability tier is associated with a first bit error rate (BER) and the second reliability tier is associated with a second BER.

3. The DNA-based storage system of claim 1, wherein the first reliability tier is associated with a first amount of parity information and the second reliability tier is associated with a second amount of parity information.

4. The DNA-based storage system of claim 1, further comprising multiple copies of the DNA strand wherein:
a first copy of the DNA strand includes a first portion associated with first data and a second portion associated with second data; and
a second copy of the DNA strand includes a first portion associated with third data and a second portion associated with the second data.

5. The DNA-based storage system of claim 1, wherein the first type of data includes data relating to storage management.

6. The DNA-based storage system of claim 1, wherein the first type of data includes data relating to a decoding process.

7. The DNA-based storage system of claim 1, wherein the first type of data has a higher level of importance when compared to the second type of data.

8. A DNA-based storage system, comprising:
an encoding system operable to:
receive data to be encoded on a DNA strand;
identify a first type of data in the received data;
identify a second type of data in the received data;
associate the first type of data with a first tier in the DNA strand; and
associate the second type of data with a second tier in the DNA strand.

9. The DNA-based storage system of claim 8, wherein the encoding system is further operable to:
associate a first amount of parity information with the first tier; and
associate a second amount of parity information with the second tier.

10. The DNA-based storage system of claim 9, wherein the second amount of parity information includes global parity information.

11. The DNA-based storage system of claim 8, further comprising a decoding system, wherein the decoding system is operable to:
generate multiple copies of the DNA strand wherein:
a first copy of the DNA strand includes a first portion associated with the first type data and a second portion associated with the second type of data; and
a second copy of the DNA strand includes a first portion associated with a third type of data and a second portion associated with the second type of data.

12. The DNA-based storage system of claim 8, wherein the first type of data has a higher level of importance when compared to the second type of data.

13. The DNA-based storage system of claim 8, wherein the first tier has a first bit error rate (BER) and the second tier has a second, higher BER.

14. A DNA-based storage system, comprising:
means for encoding data on a DNA strand, the DNA strand comprising:
a first reliability tier that is associated with a first type of data; and
a second reliability tier that is associated with a second type of data;
means for storing the DNA strand; and
means for decoding the DNA strand.

15. The DNA-based storage system of claim 14, wherein the means for encoding data on the DNA strand generates a first amount of parity information for the first reliability tier and generates a second amount of parity information for the second reliability tier.

16. The DNA-based storage system of claim 15, wherein the means for decoding utilizes the first amount of parity information when decoding data associated with the first reliability tier and uses the second amount of parity information when decoding data associated with the second reliability tier.

17. The DNA-based storage system of claim 14, wherein at least one of the first amount of parity information and the second amount of parity information includes global parity information.

18. The DNA-based storage system of claim 14, wherein the means for storing the DNA strand stores multiple copies of the DNA strand and wherein:
a first copy of the DNA strand includes a first portion associated with first data and a second portion associated with second data; and
a second copy of the DNA strand includes a first portion associated with third data and a second portion associated with the second data.

19. The DNA-based storage system of claim 18, wherein the first data and the third data are the first type of data.

20. The DNA-based storage system of claim 14, wherein the first type of data has a higher level of importance when compared to the second type of data.
